(19) United States
(12) Patent Application Publication
(10) Pub. No.: US 2022/0287772 A1
(43) Pub. Date: Sep. 15, 2022

(54) TISSUE ABLATING LASER DEVICE AND METHOD OF ABLATING A TISSUE

(71) Applicant: ADVANCED OSTEOTOMY TOOLS - AOT AG, Basel (CH)

(72) Inventors: Alfredo E. BRUNO, Biel-Benken (CH); Lorenz MARTIN, Biel (CH)

(21) Appl. No.: 17/634,307

(22) PCT Filed: Aug. 12, 2019
(86) PCT No.: PCT/EP2020/072630

§ 371 Date: Feb. 10, 2022

(30) Foreign Application Priority Data
Aug. 12, 2019 (CH) 01004/19

(51) Int. Cl. A61B 18/20

(52) U.S. Cl. CPC A61B 18/203; A61B 2018/00577

(57) ABSTRACT

A tissue ablating laser device (100) comprises a laser source (110) configured to generate a base laser beam (140) and a beam shaping optics (120) configured to receive the base laser beam (140) and to transform the base laser beam (140) to an emitting laser beam (143). The beam shaping optics (120) further is configured to focus the base laser beam (140) such that the emitting laser beam (143) has a focusing angle (142) of about 10° or less.

### TECHNICAL FIELD

The present invention relates to a tissue ablating laser device according to the preamble of independent claim 1 and more particularly to a method of ablating a tissue, for example, by means of such a tissue ablating laser device.

Tissue ablating laser devices of that kind having a laser source configured to generate a base laser beam and a beam shaping optics configured to receive the base laser beam and to transform the base laser beam to an emitting laser beam, can be used for ablating tissue in order to cut, mill or drill the tissue. The tissue can particularly be a human or animal hard tissue.

### BACKGROUND ART

For cutting and drilling various kinds of materials in many technical fields, it is increasingly popular to use apparatuses which apply a beam of laser light to ablatively remove the material instead of using mechanical tools. Indeed, nowadays, in industrial applications laser light-based cutting, milling or drilling is widespread since it allows for efficiently and flexibly processing and replicating miniature pieces at high precision as required to manufacture, e.g. in watch making, medical device production or the like. Also, in analogy, for cutting, milling and drilling human or animal hard tissue such as bones, cartilages or the like, the use of laser ablation is emerging for its various advantages. For example, in robotic surgery it is known that to use laser beams as cutting tool to replace mechanical tools results in many advantages. More particularly, e.g., in EP 2 480 153 B1 a computer assisted and robot guided laser osteotomic medical device is shown, which is based on a “cold” ablation mechanism-of-action allowing for precise and gentle drilling, milling and cutting of bone and other human or animal hard and also softer tissues.

More specifically, laser tissue ablation of biological tissue for medical purposes is being used in dermatology, urology, oncology, neurosurgery, eye surgery and other fields, where precise cutting of tissue is important. Depending on the specific purpose, different laser systems are commonly used. For example, Thulium (Tm), Holmium (Ho), Neodymium (Nd) or Erbium (Er) embedded in various solid-state glasses or crystals machined in the form of rods lasing in the infrared part of the light spectrum, which are pumped by either flash lamps or laser diodes, are used. Furthermore, CO₂ gas lasers have also been used to ablate tissue in the past. The selection of all these lasers for tissue ablation purposes is mainly due to the fact that their lasing emission wavelengths are strongly absorbed by water that is present in biological cells and, thus, in tissues. Like this, the tissues can efficiently be ablated.

Furthermore, for ablating human or animal hard tissue it has been found that when ablating certain tissues with a laser wavelength that is strongly absorbed by water, such as the Er:YAG laser having an emission line of 2.96 μm, simultaneously wetting the target hard tissue, e.g. with an aqueous solution spray, can be beneficial. For example, it has been observed that water or another liquid keeps the tissue humidified and cool during the ablation such that, beyond others, necrosis in the remaining hard tissue can be prevented.

In addition, it has been found that humidity at the target tissue may promote an indirect ablation process contributing to the overall ablation process. In this indirect ablation process, water or liquid droplets flying near the targeted tissue and/or an aqueous or liquid film condensed at the surface of the tissue can be fragmented by the electromagnetic energy of the laser pulses and propagating with kinetic energy provided by the absorbed laser beam pulses. When these small and fast travelling fragments, e.g. at velocities that could reach thousand meters per second, collide with the target tissue, the tissue is further ablated. In some cases, such indirect ablation process may be the main or even the sole contributor of the overall ablation. Particularly, when ablating hydrophobic and/or low water content tissues such as teeth or fatty tissues, the indirect ablation process often is the main or predominant tissue ablation mechanism.

Furthermore, the use of water or a similar liquid can also have another associated benefit particularly in surgical or similar medical applications. This is, it can condense the ablation debris containing some potentially toxic components that may, otherwise, contaminate the operation room.

For achieving an efficient ablation of human or animal hard tissue, typically focused laser beams are used. In particular, to have an appropriate energy density at the spot where the laser beam hits the tissue when using known beneficial laser sources, typically laser beams are provided having a focal length of 2 centimeter (cm) or less. In this connection, the energy density (ED) or flux at a beam waist (W₀) occurring at a focal point of the emitting laser beam relates to the amount of energy (E) in a single pulse divided by the diameter of the beam at W₀. (D=2 ω₀); namely, ED=F=E/D=E/2 ω₀.

Thereby, in such applications of laser sources it is important to assure that the distance to the tissue is suitable because in case the distance is too high the intensity is too low for efficiently ablating the tissue or for ablating the tissue at all. For keeping the distance constantly appropriate to achieve an intended laser beam energy density at the tissue during progressing ablation of the tissue, it is known, e.g. from EP 2 480 153 B1, to use autofocusing techniques.

However, due to the required comparably small distance to the tissue, known laser devices used for cutting, milling or drilling human or animal hard tissue typically are not suitable to satisfyingly ablate the tissue to a comparably high depth such as, e.g., a depth of more than 1 cm. Even though for many applications and treatments a depth of 1 cm is sufficient there are applications such as cutting a femur or a sternum where deeper ablation can be required.

Also, by using the laser beams having focal lengths as mentioned, typically the entrance of the hole or the cut created gets wider and wider as the depth progresses. This may result in a hole with a conical rather than a cylindrical shape or non-parallel walls in the case of a cut mimicking the focused laser beam profile. Such non-parallel or diverging opposite walls in the case of a cut may degrade the subsequent healing of the bone as the contacts between the reassembled surfaces are not ideal.

Furthermore, since in known laser devices the distance to the tissue usually is kept comparably short, the laser devices may hinder or prevent direct visualization of the intervention region. This may lead to a surgeon frequently displacing the laser device in order to be capable of direct visual inspection of the ablation process. Also, such short distance leaves no space for other instruments like retractors for soft tissue or aspirator tubes, and limits the space for the laser device positioning or the maneuverability of the laser device that can be mounted in the last stage of a robot.

Therefore, there is a need for a device, system or process allowing to cut, mill and/or drill human or animal hard tissue to comparably high depths, to create comparably parallel walls in the created cuts or holes and to improve direct visual inspection during ablation.

### DISCLOSURE OF THE INVENTION

According to the invention this need is settled by a tissue ablating laser device as it is defined by the features of independent claim 1, and by a method of ablating a tissue as it is defined by the features of independent claim 15. Preferred embodiments are subject of the dependent claims.

In one aspect, the invention is a tissue ablating laser device comprising a laser source and a beam shaping optics. The laser source is configured to generate a base laser beam. The beam shaping optics is configured to receive the base laser beam and to transform the base laser beam to an emitting laser beam. The beam shaping optics is further configured to focus the base laser beam such that the emitting laser beam has a focusing angle of about 10° or less, preferably about 5° or less and more preferably about 3° or less.

The term “laser device” generally relates to a device being arranged to generate a laser beam or which emits light through a process of optical amplification based on the stimulated emission of electromagnetic radiation. In general, laser is an acronym for “light amplification by stimulated emission of radiation”. A laser may differ from other sources of light in that it emits light coherently. Such spatial coherence can allow a laser to be focused to a tight spot, which makes applications such as cutting or lithography possible. The tissue ablating laser ablating device can particularly be a laser osteotome or configured to be used as an osteotome.

The tissue to be ablated by the laser device can be a human or animal tissue and, more particularly, a human or animal natural or artificial hard tissue. Thereby, the term “hard tissue” can relate to nail tissue, cartilage and particularly to bone tissue. Thus, the tissue ablating laser device can particularly be designed for cutting, milling and/or drilling bones. The term “artificial” in this connection can relate to a synthetic material for substituting or replacing natural hard tissue. Thus, the term “artificial hard tissue” can refer to synthetically generated tissue or materials used as bone substitutes. As mentioned above, in a particularly advantageous embodiment, the tissue ablating laser device is a bone ablating laser device or laser osteotome. More specifically, the bone ablating laser device or laser osteotome can be an automatic bone ablating laser device. For example, it can comprise a robotic unit carrying the laser source and/or the beam shaping optics, and a control unit, wherein the control unit is configured to control the robotic unit to generate a predefined cut or cutting geometry on the bone.

The beam shaping optics can comprise optical components such as lenses, telescopes, deflection or dichroic mirrors, other mirrors, collimators, reflectors, or the like. It can be embodied by a single such component or by a plurality of such components. For example, as one of the last components before the emitting laser beam is provided the beam shaping optics can comprise a focusing lens, i.e. a convex lens. The term “telescope” as used here refers to a combination of lenses, usually two lenses, a divergent lens and a convergent lens into a single element placed at a distance and used to expand a laser beam by a factor X. The distance between, or among, the lenses of the telescope can be varied so that the rays of the laser beam propagates in a parallel or almost parallel fashion.

The emitting laser beam is the laser beam emitted by the laser device. It can particularly be adapted to be directed to a target, i.e. the tissue such as a bone or other human or animal hard tissue. Thus, the emitting laser beam is the final laser beam generated by the overall laser device. Advantageously, it is adapted by the laser device to the requirements of its application or purpose.

When referring to an angle in connection with a laser beam herein, it may particularly be meant the angle of an external or outermost ray of the envelope of rays comprised by the laser beam. More specifically, the focusing angle preferably is defined by a direction of an outermost ray of the emitting laser beam and a direction of a propagation path of the emitting laser beam. The direction of the propagation path of the emitting laser beam can correspond to a direction of travel of a central ray of the emitting laser beam. The term “ray” as used herein relates to a light ray. Typically, laser beams are composed of a plurality of rays or a bundle of rays. The outermost ray of the emitting laser beam is the ray at a periphery of the beam. The rays representing the external part of the striking emitting laser beam are those having the largest focusing angle with respect to the propagation path commonly represented by a z-axis and typically defined as the center of the laser beam. The focusing angles of the internal rays are decreasing until they have 0 degrees at its center when they become co-axial with the propagation path or z-axis. Thus, since the emitting laser beam is a focused laser beam, the outermost ray is the ray having the larges focusing angle of all rays of the laser beam. The direction of the outermost ray is the direction along which this ray travels. It can also be referred to as travel direction.

The term “propagation path” as used in connection with the emitting laser beam relates to a path along which the laser beam travels after being emitted. The direction of the propagation path can correspond to an axis of the emitting laser beam or its propagation path. In particular, the direction of the propagation path can be a mean or average of all directions of the rays of the emitting laser beam.

The laser device according to the invention allows for directing the emitting laser beam at a comparably large incident angle on the tissue. Thereby, a relatively long working distance can be provided which may achieve a proper visualization and a good access to most anatomical regions such that practical recommendations and preferences suggested by surgeons can be considered.

Furthermore, the focusing angle of the emitting laser beam allows for ablating bone or other hard tissue to a depth of more than 1 cm. Like this, e.g., bones of more than 1 cm thickness can be cut. Also, the shape of the cut or hole generated by such emitting laser beam can be beneficial, e.g. having more or less parallel walls. For instance, when cutting, e.g., the sternum through the middle as required in heart or lung operations, or craniotomies of adults, both having often more than 1 cm thickness, it is important that the walls of the cut are essentially parallel for the faster post-operatory healing.

The ablation of the tissue by means of the tissue ablating laser device is based on the following: In general, tissue ablation by laser beams occurs by plural physical effects. In the most common cases, the laser light is absorbed by molecules like proteins, lipids, collagens and or other biological compounds. The conversion of absorbed laser energy leads to thermal heat resulting in a strong and rapid temperature increase. During this process, most often, molecules in the tissue are directly degraded and converted into plume or debris being ejected from an ablation spot. This can be referred to as ablation by a direct ablation process of thermal nature. Since such processes can undesirably lead to carbonization of the tissue, necrosis, precluding subsequent healing, the conditions for ablation typically have to be precisely optimized and controlled during the ablating process.

The term “plume” as used herein can relate to a product of combustion or carbonization process induced by the laser ablation and can comprise odorous molecules, smoke, aerosols and the like referred to as debris. More specifically, in the context of laser ablation, plume can summarize or comprise any substance ejected by a laser beam when hitting the target tissue as debris. The term “debris” can relate to any molecules resulting from the ablation of the target tissue such as volatile small solid fractions of the target tissue, smoke, aerosols, odorous molecules and the like.

In addition to the direct ablation, indirect ablation occurs when the tissue is wetted. Thereby, water or liquid droplets are fragmented by the electromagnetic energy of the emitting laser beam and propagated with kinetic energy provided by the absorbed laser beam. When these small and fast travelling fragments, e.g. at velocities that could reach thousand meters per second, collide with the tissue, the tissue is further ablated.

In operation of the tissue ablating laser device according to the invention, the specific focusing angle allows the emitting laser beam to strike sidewalls of an increasing cut or hole in the tissue at comparably sharp angles, i.e. 10° or below, 5° or below, or 3° or below. When the sidewalls are wetted, by hitting the sidewalls at such angle the laser beam is reflected by the water or other liquid used for wetting the tissue.

Like this, on the one hand it is prevented that the laser beam enters the tissue through the sidewalls which would ablate the sidewalls to a certain extent and, thereby, widen the cut or hole particularly at its entrance. Thus, the cut or hole can have essentially parallel sidewalls which can be beneficial for plural reasons as mentioned above.

On the other hand, reflecting the emitting laser beam at the wetted sidewalls, allows the emitting laser beam to get deeper into the cut or hole. This allows for increasing the depth of the cut or hole provided by the laser device.

More specifically, when the emitting laser beam reaches a lateral aqueous wall or a moisturized sidewall of a cut created by the tissue ablating laser device, a part of its intensity is refracted into the water or moisture according to Snell's law and absorbed therein leading to indirect ablation if the ablation threshold conditions are fulfilled as mentioned above. Depending on the thickness of the water or moisture film such refracted rays could reach the bone leading direct ablation, i.e. when the thickness of the aqueous film is very thin such as a few μm.

However, of particular importance for the present invention is the part of the remaining intensity of the emitting laser beam that is reflected on the lateral aqueous wall or sidewall which, after several reflections at points on the lateral walls can reach the bottom of the cut.

As has been found, an intensity of the reflected emitting laser beam decreases by the refraction losses according to the Fresnel equations. However, emitting laser beams with comparably small internal incident angles, i.e. the angles the emitting laser beam hits the sidewall of the cut, are associated with smaller intensity reflection losses and undergo less reflections for reaching a certain depth of the cut such as a bottom at about 4 cm or more. For example, as shown in more detail below, when the tissue ablating laser device is configured as an osteotome, hitting the sidewalls of a cut in a bone at the mentioned internal incident angles allows for providing a comparably large portion of the emitting laser beam's intensity, such as more than 50%, more than 80% or more than 90%, to the cut depth of 4 cm. Contrarily, emitting laser beams with comparably high internal incident angles undergo more numbers of reflections and are associated with higher reflection losses. Such beams will have less chances to reach a deeper region in the hole or cut. Also, refraction losses are typically lower when smaller internal incident angles are involved which might be beneficial for providing a comparably high beam intensity at the bottom of the cut.

Therefore, by providing the emitting laser beam with the focusing angle in accordance with the invention allows for efficiently achieving comparably small internal incident angles when cutting tissue such that comparably deep cuts with a proper geometry can be generated. In particular, an internal incident angle of 10° or less, of 5° or less or of 3° or less can efficiently be provided. For example, when a direction of a beam propagation path is more or less parallel to the sidewall(s) of the cut generated by the emitting laser beam, such focusing angle can efficiently and safely achieve the advantageous internal incident angles.

Summarizing the above, the tissue ablating laser device according to the invention allows to enable that a) the cutting or drilling of bone at substantial depths such as, e.g. more 1 cm, b) the working distance of the device to the anatomical region of the surgery is long enough to be of practical use for surgeons in an operation room, c) walls in the case of a cut are comparably or essentially parallel or, in the case of holes, comparably or essentially cylindrical, respectively, and, d) safety for patients, surgeons and personnel present in the operation room is increased.

Whereas for achieving the beneficial effects related to the tissue ablation of wetted or humidified tissue, wetting can be performed by any means such as, e.g., an external wetting system. However, preferably the tissue ablating laser device itself comprises a wetting equipment configured to wetting the tissue to be ablated by the emitting laser beam. More specifically, the wetting equipment can be configured to wet a cut which is generated in the tissue or bone by the emitting laser beam. In particular, an inside or a sidewall of the cut may advantageously be wetted by the wetting equipment. By including the wetting equipment into the laser device it can be assured that the tissue is appropriately wetted at the right location, i.e. where the emitting laser beam is directed to. Like this, ablation and wetting can be coordinated such that a particular efficient cutting, milling or drilling can be achieved. For wetting, a suitable liquid such as water or more specifically an aqueous solution can be used.

When the tissue ablating laser device is embodied as an automatic bone ablating laser device as described above, the wetting equipment can be configured by the control unit being configured to control the wetting equipment accordingly. In particular, the control unit can be configured to coordinate generation of a predefined cut in the bone together with the wetting of the cut.

Thereby, the wetting equipment preferably has a spray nozzle configured to generate a liquid spray to the tissue to be ablated by the emitting laser beam. Such spray nozzle allows for precisely and specifically wetting the tissue where the emitting laser beam is directed to.

For being suitable to ablate hard tissue and particularly bone tissue the cutting laser beam can have a wavelength appropriate to evaporate water or another cell component. For that purpose, the laser source preferably is configured to generate the base laser beam with a wavelength in a range of about 2.5 micrometer (μm) to about 3.5 μm, or more preferably of about 2′940 nanometer (nm). A laser source, suitable for providing such wavelengths, i.e. that are strongly absorbed by water, can be an Er:YAG laser having an emission line of 2.94 μm. By, in addition, simultaneously wetting the hard tissue with the aqueous solution, the ablation rate can be highly increased.

The laser sources advantageously employed for ablation are pulsed lasers rather than continuous wave lasers for various reasons. The term “laser pulse” as used herein can relate to a comparably short-time laser beam preferably of a given wavelength having a specific temporal width, shape and/or power. One benefit of pulsed lasers is related to the fact that to induce ablation, the peak energy of the pulses of laser light, within a given volume and irradiation time, has to surpass a given minimum ablation threshold otherwise the deposited energy in the tissue is mainly converted into heat rather than in ejected debris as intended. However, if the fluence (F) or energy density (ED) is too high while the cooling, provided for example by the water solution and/or the intrinsic heat dissipation provided by heat diffusion and/or circulating blood in the surrounding tissue, do not suffice to control the temperature raise at the surfaces of the cut, the remaining tissue gets warmer and warmer with each subsequent pulse and, at some point, it starts to carbonize leading to undesirable necrosis that precludes the subsequent healing. In other words, the laser beam pulses of a given wavelength, including its repetition rate, pulse energy and density, pulse width, beam focusing characteristics, etc., required for the effective “cold” ablation of tissues, supported by the humidifying and cooling by a liquid, can be optimized and controlled to have an efficient and free of necrosis ablation rate. Therefore, the laser source preferably is configured to generate the base laser beam as pulsed laser beam. More specifically, the laser source preferably is configured to generate the pulsed base laser beam at a frequency in a range of about 1 to about 100 Hertz or in a range of about 10 to about 30 Hertz. Such frequency ranges allow for appropriately conditioning the tissue or ablation such that sufficient cooling and a high ablating efficiency can be achieved.

Another parameter of relevance in laser ablation is not only the energy per pulse but the temporal width of the pulses that determines the peak power. For the typical cases mentioned above of, e.g., a pulse of the free running Er:YAG or Nd:YAG lasers having, e.g., 1J of energy spread in 200 μs the peak power amounts to 5 kW whereas for the Q-switched Nd:YAG laser having e.g. 100 mJ spread in 15 ns the peak power is 6.7 MW which is thousands time higher than when the same laser is run in free running mode. It is also important to compare these values with those of a continuous wave (cw) laser operated at 10 W with very low peak power of also 10W explaining the fact that cw lasers are not suitable for laser ablation. Thus, the laser source preferably is configured to generate pulses of the pulsed base laser beam having a temporal width in a range of about 5 microseconds (μs) to about 300 μs such as 200 μs or 250 μs, in a range of about 10 μs to about 150 μs, or in a range of about 50 μs to about 120 μs.

Preferably, the beam shaping optics is configured to generate the emitting laser beam with a focal length (FL) of more than about 4 centimeter (cm). Thereby, the FL of the emitting laser beam is less than about 25 cm or less than about 20 cm. Focal length, as used here, for a single optical element such as the beam shaping optics, comprising a single lens or a combination of lenses assembled into the single optical element, which can relate to a distance between the exit of the single optical element of beam shaping optics to a waist or focal point of the focused emitting laser beam.

In this context, the F-number (F/#), as used here, is a dimensionless number denoting the ratio of the FL to a diameter (D) of the emitting laser beam striking the beam shaping optics; i.e. F/#=FL/D. For example, for an emitting laser beam of D=10 mm, and a lens with a FL=150 mm, F/#=15.

Preferably, the tissue ablating laser device is configured such that the emitting laser beam has a beam quality factor M squared (M²) of about 15 or less or of about 10 or less. In laser science, the beam quality factor represented by the parameter M² indicates the degree of variation of a beam from an ideal Gaussian beam. It can be calculated from the ratio of the beam parameter product of the beam to that of a Gaussian beam with the same wavelength. Typically, it relates to the beam divergence of a laser beam relative to the minimum focused spot size that can be achieved. For a single mode (Gaussian) laser beam, M² is exactly one. The parameter M² of a laser beam is widely used in laser science and industry as a specification parameter and its method of measurement is regulated as a standard of the International Organization for Standardization, i.e. ISO Standard 11146, 2005.

In addition to the beam quality factor or beam propagation ratio M² also another beam quality factor K is used to evaluate the quality of a given laser based on the divergence characteristics of the emitted laser beam. The two parameters K and M² depend from each other and can be calculated by using the experimentally measured beam parameters. The two parameters can be defined as

$${M}^{2} = \frac{1}{K} = \frac{π {ω}_{0} Θ}{λ}$$

where

ω₀: focal spot radius (half the beam waist D)
θ: half opening angle in the far field for the divergence
λ: wavelength
Kₘₐₓ=M²ₘᵢₙ=1.

More specifically, both parameters M² and K can describe how close an actual non-Gaussian beam compares to a perfect Gaussian beam. M²=1=K would correspond to a perfect Gaussian beam that is practically impossible to achieve. However, the smaller M² is, the better the laser beam quality is which results in a smaller divergence and thus a smelled beam diameter at the beam waist.

For achieving a M² in the preferred range, the laser source and the beam adjusting optics are appropriately chosen and adjusted. By having such M², the emitting laser beam can particularly be beneficial for and suitable to be reflected by humidity at a sidewall of a cut such that the power or beam intensity reaching a comparably high depth can be comparably high which allows to efficiently achieve a comparably deep cut.

In another aspect, the invention is a method of ablating a tissue such as a human or animal hard tissue and in particular a bone tissue or bone. The method comprises: generating a focused emitting laser beam, and directing the focused emitting laser beam to a surface of the tissue such that an internal incident angle of the emitting laser beam in relation to the tissue is about 10° or less, preferably about 5° or less and more preferably about 3° or less. The incident angle is an angle between the emitting laser beam and a surface of the tissue where it is hit by the emitting laser beam. Thereby, in progressing ablation of the tissue a cut is created which can have any desired shape. At least initially, the cut typically is a hole. Usually, such cut has a sidewall and a bottom. The sidewall of the cut or hole can be essentially perpendicular to a plane defined by a surface of the tissue.

Thereby, internal the incident angle preferably is defined by a direction of an outermost ray of the emitting laser beam and the sidewall of the cut in the tissue.

As the emitting laser beam hits the sidewall at an angle of about 10° or less, the effects and benefits described above in connection with the tissue ablation laser device according to the invention can efficiently be achieved.

By means of the following preferred embodiments of method according to the invention, the effects and benefits described above in connection with the preferred embodiments of the tissue ablation laser device according to the invention can efficiently be achieved.

Preferably, the method comprises a step of wetting the tissue to be ablated by the emitting laser beam. Thereby, the liquid preferably is sprayed to the tissue to be ablated by the emitting laser beam.

Wetting the tissue to be ablated by the emitting laser beam preferably comprises wetting the sidewall of the cut in the tissue. Like this, a particularly efficient reflection in the cut can be achieved which may increase depth of field (DOF) and allows for generating a higher cut depth in the tissue or bone.

Preferably, the emitting laser beam is generated at a wavelength in a range of about 2.5 μm to about 3.5 μm, or of about 2′940 nm.

Preferably, the emitting laser beam is generated as pulsed laser beam. Thereby, the emitting base laser beam preferably is pulsed at a frequency in a range of about 1 to about 500 Hertz or in a range of about 10 to about 30 Hertz. Pulses of the pulsed base laser beam preferably have a temporal width in a range of about 5 μs to about 300 μs such as 200 μs or 250 μs, in a range of about 10 μs to about 150 μs, or in a range of about 50 μs to about 120 μs.

Preferably, the emitting laser beam is generated with a focal length of more than about 4 cm. Thereby, the focal length of the emitting laser beam preferably is less than about 25 cm or less than about 20 cm.

Preferably, the method according to the invention is not a method for treatment of the human or animal body by surgery or therapy. Such ex vivo or in vitro method can be beneficial in many applications. In particular, when being such ex vivo method no surgical step is involved but the cutting is performed completely outside and distant from any living human or animal body.

### BRIEF DESCRIPTION OF THE DRAWINGS

The tissue ablating laser device according to the invention and the method according to the invention are described in more detail herein below by way of an exemplary embodiment and with reference to the attached drawings, in which:

FIG. 1 shows a schematic view of a first embodiment of a tissue ablating laser device according to the invention;

FIG. 2 shows a schematic view of a second embodiment of a tissue ablating laser device according to the invention; and

FIG. 3 shows a graph of fractions of intensity at a bottom of a cut depending on varying internal incident angles.

### DESCRIPTION OF EMBODIMENTS

In the following description certain terms may be used for reasons of convenience and are not intended to limit the invention. The terms “right”, “left”, “up”, “down”, “under” and “above” refer to directions in the figures. The terminology comprises the explicitly mentioned terms as well as their derivations and terms with a similar meaning. Also, spatially relative terms, such as “beneath”, “below”, “lower”, “above”, “upper”, “proximal”, “distal”, and the like, may be used to describe one element's or feature's relationship to another element or feature as illustrated in the figures. These spatially relative terms are intended to encompass different positions and orientations of the devices in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. The devices may be otherwise oriented (rotated 90 degrees or at other orientations), and the spatially relative descriptors used herein interpreted accordingly. Likewise, descriptions of movement along and around various axes include various special device positions and orientations.

To avoid repetition in the figures and the descriptions of the various aspects and illustrative embodiments, it should be understood that many features are common to many aspects and embodiments. Omission of an aspect from a description or figure does not imply that the aspect is missing from embodiments that incorporate that aspect. Instead, the aspect may have been omitted for clarity and to avoid prolix description. In this context, the following applies to the rest of this description: If, in order to clarify the drawings, a figure contains reference signs which are not explained in the directly associated part of the description, then it is referred to previous or following description sections. Further, for reason of lucidity, if in a drawing not all features of a part are provided with reference signs it is referred to other drawings showing the same part. Like numbers in two or more figures represent the same or similar elements.

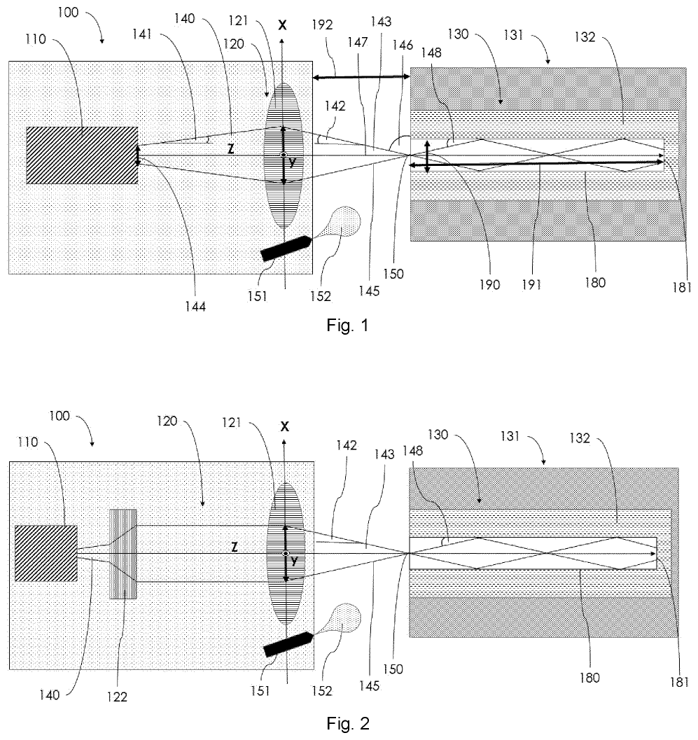

FIG. 1 shows a first embodiment of a tissue ablating laser device 100 operated in accordance to apply an embodiment of a method according to the invention. The tissue ablating laser device 100 comprises a laser source 110 and a beam shaping optics 120. The laser source 110 is an Er:YAG laser having an emission line of 2.96 μm and generating a pulsed base laser beam 140 at a frequency of about 20 Hertz and with a temporal width of about 80 μs. The base laser beam 140 starts with an initial diameter 144 and widens at a diverging angle 141.

The beam shaping optics 120 comprises a convex focusing lens 121. The laser source 110 and the focusing lens 121 are positioned and oriented such that the base laser beam 140 is received by a left hand entry side of the lens 121. The lens 121 transforms the laser beam and emits it in form of an emitting laser beam 143 from a right hand side along a z-axis which corresponds to a propagation path or propagation direction.

The lens 121 focuses the base laser beam 140 such that the emitting laser beam 143 has a focal point 150 at a focal length of 15 cm. Moreover, the lens 121 shapes the emitting laser beam to have a focusing angle 142 of 6°. The focusing angle 142 is defined by a direction of an outermost ray 145 of the emitting laser beam 143 and the direction of the propagation path of the emitting laser beam 143, i.e. the z-axis or the direction of a central ray 147.

The tissue ablating laser device 100 further comprises a spray nozzle 151 as wetting equipment. The spray nozzle 151 is configured to generate a liquid spray 152 wherein the liquid particularly is an aqueous solution.

In operation of the tissue ablating laser device 100 to embody the method according to the invention, the tissue ablating laser device 100 is positioned at a distance of about 12 cm to a bone 131 as tissue to be ablated. More specifically, the tissue ablating laser device 100 is oriented such that the emitting laser beam 143 and the spray 152 are directed to a location of the bone 131 to be ablated. Thereby, the emitting laser beam 143 generates a cut 130 which increases in depth 191 while the emitting laser beam 143 is provided. The cut 131 has a diameter 190 defined by an energy density (ED) or flux of the emitting laser beam 143.

The spray 152 creates an aqueous film 132 inside the cut 130, wherein sidewalls 180 and a bottom 181 of the cut 130 are covered by the aqueous film 132. Since the emitting laser beam 143 has the focusing angle 142 of 6°, i.e. below 10°, light or rays of the emitting laser beam 143 strikes the sidewalls 180 of the cut 130 at a comparably sharp angle. This effectuates that a comparably large extent of the light of the emitting laser beam 143 is reflected at the sidewalls 180 such that a considerable portion advances to the bottom 181 of the cut 130. Like this, the bottom 181 is ablated and a comparably large depth such as 4 cm or more can be cut into the bone 131.

Thus, by means of the correctly positioned and oriented tissue ablating laser device 100 the focused emitting laser beam 143 is generated and directed to an outer surface of the bone 131 such at an external incident or initial angle 146 towards an outside plane of the surface of the bone 131 of about 94°. When advancing ablation, the cut 130 is created. Since the emitting laser beam 143 is directed orthogonal to the outer surface of the bone 131, the sidewalls 180 extend at about 90° from the outer surface of the bone 131. Thereby, the emitting laser beam 143 hits the sidewalls 180 at an internal incident angle 148 of about 6°. The internal incident angle 148 is defined by a direction of an outermost ray 145 of the emitting laser beam 143 and the sidewall 180 of the cut 130 in the bone.

In FIG. 2 a second embodiment of a tissue ablating laser device 100 is shown, which is widely identically designed and operated as the first tissue ablating laser device 100 of FIG. 1. The only difference is that the ablating laser device 100 is equipped with a different beam shaping optics 120. In particular, the beam shaping optics 120 has a focusing lens 121 and, in addition, a collimating lens 122. The collimation lens 122 is positioned upstream of the base laser beam 140 relative to the focusing lens 121. More specifically, the collimating lens 122 receives the base laser beam 140 generated by the laser source 110, collimates the light and provides the collimated beam to the focusing lens 121. The focusing lens then focuses the light and provides the emitting laser beam 143 as described above.

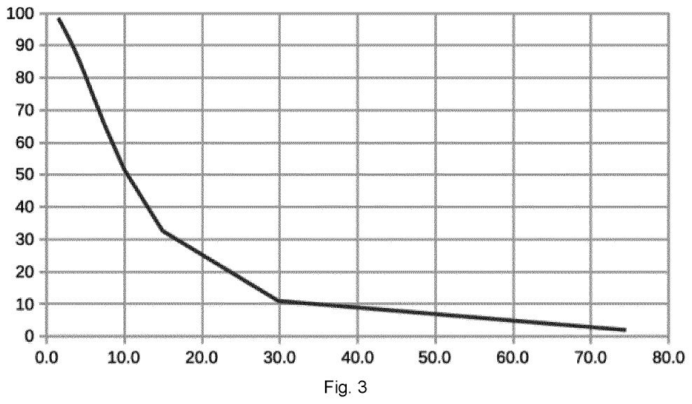

FIG. 3 shows a graph of a fraction in percent of beam intensity or power of the emitting laser beam 143 reaching the bottom 181 of the cut 130 generated in the bone 131. The axis of ordinates represents a percentage of the fraction of the beam intensity of the emitting laser beam 143 at the bottom 181 of the cut 130. The cut 130 is a hole having a depth of 4 cm and a diameter of 1 cm. The axis of abscissas represents the internal incident angle 148 in degrees. The emitting laser beam 143 has a wavelength A of 2,96 μm and a diameter of 1.3 mm.

As can be seen in FIG. 3, when providing the emitting laser beam 143 particularly suitable for cutting bone, the fraction of the intensity at the bottom 181 of the 4 cm deep cut 130 in the bone 131 still is more than 50%, when the internal incident angle is 10°. Such cut depth is sufficient for cutting most of human bones. Thus, since the fraction of the intensity is more than 50% the bone 131 can still be efficiently cut even at this comparably high depth.

However, as can be seen in the graph of FIG. 3, the fraction of the intensity at the bottom 181 of the cut rapidly increases when lowering the internal incident angle 148 below 10°. Thus, by a comparably small adaptation of the internal incident angle 148 a considerable increase of ablation efficiency can be achieved. For example, by lowering the internal incident angle 148 to about 5° about 80% of the emitting laser beam intensity arrives the bottom of the cut 130. Or, at an internal incident angle of about 3° even about 90% of the emitting laser beam intensity can be provided to the bottom of the cut 130.

This description and the accompanying drawings that illustrate aspects and embodiments of the present invention should not be taken as limiting-the claims defining the protected invention. In other words, while the invention has been illustrated and described in detail in the drawings and foregoing description, such illustration and description are to be considered illustrative or exemplary and not restrictive. Various mechanical, compositional, structural, electrical, and operational changes may be made without departing from the spirit and scope of this description and the claims. In some instances, well-known circuits, structures and techniques have not been shown in detail in order not to obscure the invention. Thus, it will be understood that changes and modifications may be made by those of ordinary skill within the scope and spirit of the following claims. In particular, the present invention covers further embodiments with any combination of features from different embodiments described above and below.

The disclosure also covers all further features shown in the Figs. individually although they may not have been described in the afore or following description. Also, single alternatives of the embodiments described in the figures and the description and single alternatives of features thereof can be disclaimed from the subject matter of the invention or from disclosed subject matter. The disclosure comprises subject matter consisting of the features defined in the claims or the exemplary embodiments as well as subject matter comprising said features.

Furthermore, in the claims the word “comprising” does not exclude other elements or steps, and the indefinite article “a” or “an” does not exclude a plurality. A single unit or step may fulfil the functions of several features recited in the claims. The mere fact that certain measures are recited in mutually different dependent claims does not indicate that a combination of these measures cannot be used to advantage. The terms “essentially”, “about”, “approximately” and the like in connection with an attribute or a value particularly also define exactly the attribute or exactly the value, respectively. The term “about” in the context of a given numerate value or range refers to a value or range that is, e.g., within 20%, within 10%, within 5%, or within 2% of the given value or range. Components described as coupled or connected may be electrically or mechanically directly coupled, or they may be indirectly coupled via one or more intermediate components. Any reference signs in the claims should not be construed as limiting the scope.

1.-26. (canceled)

27. A tissue ablating laser device comprising:
a laser source configured to generate a base laser beam; and
a beam shaping optics configured to receive the base laser beam and to transform the base laser beam to an emitting laser beam,
wherein the beam shaping optics is configured to focus the base laser beam such that the emitting laser beam has a focusing angle of about 10° or less, of about 5° or less, or of about 3° or less.

28. The tissue ablating laser device of claim 27, wherein the focusing angle is defined by a direction of an outermost ray of the emitting laser beam and a direction of a propagation path of the emitting laser beam.

29. The tissue ablating laser device of claim 28, wherein the direction of the propagation path of the emitting laser beam corresponds to a direction of a central ray of the emitting laser beam.

30. The tissue ablating laser device of claim 27, comprising a wetting equipment configured to wet the tissue to be ablated by the emitting laser beam, wherein the wetting equipment preferably is configured to wet a cut generated in the tissue by the emitting laser beam.

31. The tissue ablating laser device of claim 30, wherein the wetting equipment has a spray nozzle configured to generate a liquid spray to the tissue to be ablated by the emitting laser beam.

32. The tissue ablating laser device of claim 27, wherein the laser source is configured to generate the base laser beam with a wavelength in a range of about 2.5 micrometer to about 3.5 micrometer, or of about 2′940 nanometer.

33. The tissue ablating laser device of claim 27, wherein the laser source is configured to generate the base laser beam as a pulsed laser beam.

34. The tissue ablating laser device of claim 33, wherein the laser source is configured to generate the pulsed base laser beam at a frequency in a range of about 1 to about 100 Hertz or in a range of about 10 to about 30 Hertz, and/or
pulses of the pulsed base laser beam having a temporal width in a range of about 5 microseconds to about 300 microseconds, in a range of about 10 microseconds to about 150 microseconds, or in a range of about 50 microseconds to about 120 microseconds.

35. The tissue ablating laser device of claim 27, wherein the beam shaping optics is configured to generate the emitting laser beam with a focal length of more than about 4 cm, wherein the focal length of the emitting laser beam preferably is less than about 25 cm or less than about 20 cm.

36. The tissue ablating laser device of claim 27 being configured to be applied as an osteotome.

37. The tissue ablating laser device of claim 27 configured such that the emitting laser beam has a beam quality factor M squared of about 15 or less or of about 10 or less.

38. A method of ablating a tissue such as a human or animal hard tissue, comprising:
generating a focused emitting laser beam; and
directing the focused emitting laser beam to a surface of the tissue such that an internal incident angle of the emitting laser beam in relation to the tissue is about 10° or less, preferably about 5° or less and more preferably about 3° or less.

39. The method of claim 38, wherein the internal incident angle is defined by a direction of an outermost ray of the emitting laser beam and a sidewall of a cut in the tissue.

40. The method of claim 38, comprising a step of wetting the tissue to be ablated by the emitting laser beam.

41. The method of claim 40, wherein wetting the tissue to be ablated by the emitting laser beam comprises wetting the sidewall of the cut in the tissue.

42. The method of claim 40, wherein a liquid is sprayed to the tissue to be ablated by the emitting laser beam.

43. The method of claim 38, wherein the emitting laser beam is generated at a wavelength in a range of about 2.5 micrometer to about 3.5 micrometer, or of about 2′940 nanometer.

44. The method of claim 38, wherein the emitting laser beam is generated as a pulsed laser beam, wherein the emitting base laser beam preferably is pulsed at a frequency in a range of about 1 to about 100 Hertz or in a range of about 10 to about 30 Hertz.

45. The method of claim 44, wherein pulses of the pulsed base laser beam have a temporal width in a range of about 5 microseconds to about 300 microseconds, in a range of about 10 microseconds to about 150 microseconds, or in a range of about 50 microseconds to about 120 microseconds.

46. The method of claim 38, wherein the emitting laser beam is generated with a focal length of more than about 4 cm, wherein the focal length of the emitting laser beam preferably is less than about 25 cm or less than about 20 cm.
